(19) United States
(12) Patent Application Publication
(10) Pub. No.: US 2017/0333503 A1
(43) Pub. Date: Nov. 23, 2017

(54) Process for extraction, separation and purification of cannabinoids, flavonoids and terpenes from cannabis

(71) Applicant: Total Health care I, Rockford, IL (US)

(72) Inventor: David Ayres, Rockford, IL (US)

(73) Assignee: Total Health care I, Rockford, IL (US)

(21) Appl. No.: 14/999,664

(22) Filed: Jun. 9, 2016

Related U.S. Application Data
(60) Provisional application No. 62/173,197, filed on Jun. 9, 2015.

(51) Int. Cl. A61K 36/185; A61K 31/465; A61K 36/81

(52) U.S. Cl. CPC A61K 36/185; A61K 36/81; A61K 31/465; A61K 2236/33; A61K 2236/15

(57) ABSTRACT

A method of extracting natural products, such as cannabinoids, flavonoids and terpenes, from plant material such as a Cannabis genus of plant is disclosed. The method affords a quick and efficient extraction of natural products without further purification.

This application claims priority to and the benefit of application Ser. No. 62/173,197 filed on Jun. 9, 2015, the entirety of which is incorporated herein by reference.

### FIELD OF THE INVENTION

This invention generally relates to a method of extracting natural products from a plant or plant material. In a specific example, the invention relates to a method of extracting cannabinoids, flavonoids and terpenes from a Cannabis genus of plant.

### BACKGROUND OF THE INVENTION

Safe medical use of cannabinoids ultimately requires extraction and purification from a Cannabis plant in order to give the proper measured dosage of the purified and analyzed active pharmaceutical ingredient, or medicine, as prescribed by the physician. The therapeutic activity of plant medicines is attributed to the active constituents that they contain. In some cases the intrinsic activity of natural products has been linked to specific chemical species, but in other cases the activity of the plant medicine is considered to be due to a combination of constituents acting in concert. In most plant materials, the active constituent is present in varying proportions. In the case of cannabis resin, the concentration of active constituent may be more than 60% W/W of resin (hashish). Whatever the concentration in biomass, it is convenient to extract specific constituents, or produce an enriched extract, which can be then formulated into conventional dosage forms for ease of administration.

The principle cannabinoid components present in herbal Cannabis are the cannabinoid acids Δ⁹tetrahydrocannabinolic acid (Δ⁹THCA) and cannabidiolic acid (CBDA), with small amounts of the corresponding neutral cannabinoids, respectively Δ⁹ tetrahydrocannabinol (Δ⁹THC) and cannabidiol (CBD). In addition to these major cannabinoids, herbal Cannabis may contain lower levels of other minor cannabinoids. Examples of other cannabinoids include cannabigerol (CBG), cannabichromene (CBC), and Δ⁹ tetrahydrocannabivarin (Δ⁹THCV).

With the advent of legalizing the medicinal use of cannabis in many states, the extraction of medicinal compounds from raw Cannabis plant in large volumes is in demand. The most significant driver for this demand is the increasing bodies of research showing tangible benefits to individuals suffering from a plurality of ailments when using medicinal cannabis. Furthermore no toxic or overdose effects from the use of cannabis have been medically documented.

To this end, Illinois Senate Bill 2636 allows for patients under 18 years of age with epilepsy to treat the seizures under medical supervision with only medical infused marijuana products, which requires a safe and completely non-toxic method of extraction. It is also preferable to offer younger patients nonpsychoactive (and even anti-psychotic) cannibinoids such as cannabidiol (CBD), with the psychoactive components present in cannabis removed. As noted by Hampson et al. (U.S. Pat. No. 6,630,507), the therapeutic potential of nonpsychoactive cannabinoids is particularly promising, because of the absence of psychotoxicity, and the ability to administer higher doses than with psychotropic cannabinoids, such as THC. Previous studies also indicated that cannabidiol is not toxic, even when chronically administered to humans or given in large acute doses (700 mg/day). The treatment of brain tumors requires a 1:1 to 1:20 w/w ratio of THC to CBD (Ross et al., U.S. Pat. No. 8,481,091). This also requires a safe and non-toxic method of extraction to get the medicines to this ratio.

Cannabinoid-containing plant extracts may be obtained by various means of extraction of cannabis plant material. Such means include but are not limited to: supercritical or subcritical extraction with carbon dioxide (CO₂), extraction with hot gas and extraction with polar solvents. These methods, which are known by those skilled in the art, are deficient for many reasons.

For example, the disadvantage of extraction with non-polar solvents is that they are derived from petroleum and are highly flammable, even in closed systems, as they tend to create static when mixed. They also contain trace amounts of heavy metals and other toxins, which will be concentrated in the medicine after removing the solvent. Non-polar solvents are also much more toxic than polar solvents. Many non-polar solvents are generally known to cause cancer, and they will also remain in the extract in trace amounts even after vacuum removal. Also, a process involving extraction with non-polar solvents only produces an extract that is 30-70% pure THC, the balance being undesirable waxes, resins and other material that are removed via vacuum distillation. Vacuum distillation also removes the desirable flavonoids and terpenes. In addition, the extraction is only done in small batches (e.g. 2 lbs. of plant material) because it becomes even less efficient on a larger scale. In short, this method of extraction only produces 3-10% THC by weight of the plant material (U.S. Pat. No. 6,365,416).

One problem with the supercritical carbon dioxide extraction is that the cost of the equipment makes it cost-prohibitive. It is also done under high pressure (2000-5000 psig, or pounds per square inch gage), which is generally detrimental to the quality and yield of the final product extract. The method also produces low purity extract (30-70% pure THC, 3-10% by weight of the plant) (Murty et al., US Publication No. 2003/0050334) and there is also a significant amount of water that binds to the THC. Another drawback to supercritical CO₂ extraction is that it is performed in small batches. Each batch can take up to eight hours to complete. Also, and similar to non-polar solvent extraction methods, the extract is further purified by vacuum.

Hot gas extraction, while affording pure samples of THC content, however overall yields are generally low. Extraction via heated gas also requires expensive equipment. In addition, elevated temperatures are obviously needed along with gas, a combination leading to potential degradation of the natural product either via pyrolysis or oxidation (U.S. Pat. No. 7,622,140).

A cheaper and safer extraction of cannabinoid from a Cannabis genus of plant is desired. The method ideally produces a high overall yield of extract, with a purity of cannabinoids above 90%, the balance being flavonoids and terpenes. It is further desired that the extract contain substantially no waxes, resins or other undesirable compounds.

### BRIEF SUMMARY OF THE INVENTION

In accordance with a first aspect of the invention there is provided a process for preparing an extract from a plant or plant material, for example a Cannabis genus of plant, which comprises treating the plant or plant material with at least one polar solvent and optionally carbon dioxide as co-solvent at a temperature of between −25° C. to −100° C.

Other aspects, objectives and advantages of the invention will become more apparent from the following detailed description. While the invention will be described in connection with certain preferred embodiments, there is no intent to limit it to those embodiments. On the contrary, the intent is to cover all alternatives, modifications and equivalents as included within the spirit and scope of the invention as defined by the appended claims.

### BRIEF DESCRIPTION OF THE DRAWINGS

FIG. 1 shows GC/MS results of an extract from marijuana plant material 1 using the extraction method disclosed herein.

FIG. 2 is an area percent report of the GC/MS of FIG. 1.

FIG. 3 shows GC/MS results of an extract from marijuana plant material 2 using the extraction method disclosed herein.

FIG. 4 is an area percent report of the GC/MS of FIG. 3.

FIG. 5 shows GC/MS results of an extract from a wild hemp plant material using the extraction method disclosed herein.

FIG. 6 is an area percent report of the GC/MS of FIG. 5.

FIG. 7 shows GC results of an extract from marijuana plant material 3 using the extraction method disclosed herein.

FIG. 8 shows GC/MS results of the residual solvents present in the extract of marijuana plant 3.

FIG. 9 shows GC-UV results (cannabinoids) of an extract from marijuana plant material 4 using the extraction method disclosed herein.

FIG. 10 shows GC-UV results (terpenes) of an extract from marijuana plant material 4 using the extraction method disclosed herein.

FIG. 11 shows GC/MS results of the pesticides and residual solvents present in the extract of marijuana plant material 4.

FIG. 12 shows GC/MS results of the residual solvents present in the extract of marijuana plant material 4.

### DETAILED DESCRIPTION OF THE INVENTION

Polar solvents, many of which are available in “food-grade” quality, provide herein a more optimal extraction for natural product medicines that can be consumed by patients. Polar solvents are commonly used for extracting flavorings for food and enzymes for cheese production. Polar solvents have also been used in extraction of natural products, but usually in conjunction with other methods (see U.S. Pat. Nos. 8,895,078; 8,846,409; and 7,622,140; all incorporated herein by reference). Polar solvents are preferably low molecular weight solvents and include but are not limited to ethanol, methanol, isopropanol, methyl ethyl ketone, acetone, acetonitrile. More preferably, the at least one polar solvent is selected from the group consisting of ethanol, isopropanol and acetone. Generally, the polar solvent is itself soluble in water.

According to an embodiment of the invention, polar, water miscible solvents combined with dissolved non-polar CO₂ at low temperatures (−25° C. to −100° C.) and atmospheric pressure allow more selective extraction and purification of cannabinoids. In addition, the methods of the invention provide for a gentler and more complete extraction. The extraction is not done under pressure and the cold temperatures greatly reduce flammability or explosion potential by lowering the vapor pressure of the solvent below its flash point. The extraction is also blanketed by the evaporating CO₂, which removes the oxygen and reduces or eliminates the flammability risks. Since they are water miscible, polar solvents also offer the possibility of further purification by recrystallization, which non-polar solvents do not.

Thus, in accordance with embodiments of the invention, and to overcome the deficiencies of the prior art, a process for preparing an extract from a plant or plant material, for example a Cannabis genus of plant, which comprises treating the plant or plant material with at least one polar solvent and optionally carbon dioxide as co-solvent at a temperature of between −25° C. to −100° C. is provided. Another example of preparing an extract from a plant or plant material is extracting a nicotine extract from a tobacco plant. In a preferred embodiment, the temperature of the extraction occurs at between −35° C. to −90° C., more preferably −40° C. to −80° C., even more preferably −50° C. to −80° C., and most preferably between −60° C. to −70° C.

It is believed that dissolved CO₂ in a polar solvent makes it both polar and non-polar in nature allowing for a more selective and complete extraction, as cannabinoids have both polar and non-polar sites on the molecules. Dissolving CO₂ in the water miscible polar solvents also provides the very low and controllable temperatures at atmospheric pressure that most selectively extract the desired cannabinoids, flavonoids, and terpenes.

It is also believed that both the cold temperatures and the dual polar/non-polar solvent system are the reasons that the waxes and resins are not extracted out. The physical agitation of the CO₂ bubbles is another possible reason the desired materials from the plant material are removed for complete selectivity. The CO₂ as used in the instant invention seems to have all of the benefits of the supercritical CO₂ extraction methods with none of the drawbacks. This includes the high overall yield of around 20%, and the overall purity of the yield of over 90 weight percent cannabinoids.

CO₂ is highly soluble (up to 50% w/w and v/v) in most very cold polar solvents. The extraction method of the present invention is performed at a very cold temperature (most preferably at least between −60° C.-70° C.) in a solvent system that is 20-70% CO₂/80-30% polar solvent, and preferably 40-60% CO₂/60-40% polar solvent, and more preferably about 50% polar solvent (acetone) and about 50% CO₂.

The reason this dual solvent system is so selective for cannabinoids is that it is Δ⁹tetrahydrocannabinolic acid (Δ⁹THCA) and cannabidiolic acid (CBDA) that is predominantly present in the plant, and not Δ⁹ tetrahydrocannabinol (Δ⁹THC) and cannabidiol (CBD). THCA and CBDA are both water-soluble and have both polar and non-polar sites on the molecule.

In an embodiment, the extraction is complete in about 30 minutes or less and can be scaled up very easily and safely. Also, the fact that the material stays in a liquid state throughout the whole process means that on a larger scale, it can be done in an automated closed system.

In still another embodiment, the extract is optionally subjected to a decarboxylation step. The purpose of the decarboxylation step is to convert cannabinoid acids present in the plant material to the corresponding free cannabinoids. By “cannabinoid acid” is meant a cannabinoid having at least one carboxylic acid moiety as part of the molecule, wherein by “free cannabinoid” is meant a cannabinoid with no carboxylic acid moiety as part of the molecule. Decarboxylation is preferably carried out by heating the plant material to a defined temperature (over 100° C. and typically less than 150° C.) for a suitable length of time. Decarboxylation of cannabinoid acids is a function of time and temperature, thus at higher temperatures a shorter period of time is taken for complete decarboxylation of a given amount of cannabinoid acid. In selecting appropriate conditions for decarboxylation, consideration must, however, be given to minimizing thermal degradation of the desirable, pharmacological cannabinoids into undesirable degradation products, particularly thermal degradation of Δ⁹THC to cannabinol (CBN).

In therefore another embodiment, a first step includes maceration of the plant material. Extraction with the dual polar solvent/CO₂ system is then performed at decreased temperatures as described above. In a preferred embodiment, 200 ml of acetone is used per 28 grams of plant material with enough CO₂ to maintain a solvent system of about 50/50 weight percent acetone/CO₂ at a temperature of between −60° C. to −70° C. It is noted that, generally, CO₂ can be added throughout the extraction as it evaporates. Once the extraction is complete (generally not longer than 30 minutes), the solvent with dissolved extract is filtered from plant material. The solvent is then removed under vacuum. The extract is then decarboxylated at 104-120° C. to afford an extract that is consistently >90% THC/CBD with no need for further purification. The balance of the extract (5-10%) is the desired flavonoids and terpenes. Waxes and resins are substantially absent from the extract by the process described herein. As disclosed herein, the method of the invention is capable of extracting 20% THC/CBD by weight from the plant material, compared to 3-10% by the other methods.

As can be seen in the Examples below, the overall yield is reflected in the quality of the plant material. Generally, however, with the use of high quality plant material, the overall yield of the extract when utilizing the method disclosed herein is between 15-20%, and preferably between 18-20%, with respect to the total weight of the plant material. In addition, the disclosed method affords an extract that consists essentially of desired natural products, such as for example, cannabinoids, flavonoids and terpenes in the case of marijuana or similar-type plant material. Thus, no additional purification step is necessary.

In yet another embodiment, the polar, water miscible solvents is recovered and recycled, which is more environmentally friendly than using non-polar solvents.

### EXAMPLES

The extract is weighed and analyzed by GC/MS and recorded. 2-4 pounds of Cannabis plant material is acquired based on the assumption that the cannabinoid content is approximately 10% by weight of plant material. The plant material can be purchased from the University of Mississippi or a federally licensed hemp cultivation facility. Alternatively, a DEA import permit is required, since there are sources for organic hemp in Canada.

### Example 1—General Method

In a blender, 1 ounce of plant material is grinded into a fine powder. In a 500 ml beaker on magnetic stir plate, 200 ml of acetone is added. To the acetone, dry ice is slowly added until the temperature reaches about −70° C. and about 50% CO₂ w/w and v/v is reached relative to the acetone. The ground plant material is then added to the solvent system and stirred for 30 minutes, with continuous addition of dry ice to maintain −70° C. and 50% w/w and v/v liquid CO₂. After about 30 minutes, the plant material is filtered out. The resulting solution is then warmed gently in a rotary evaporator at 40° C. for a few minutes to remove the CO₂. Once the CO₂ is removed, vacuum is applied to remove the acetone. The concentrated extract is then placed in a small distillation apparatus to remove any residual acetone, followed by heating the extract to 104° C. to decarboxylate the cannabinoids. The product is found to be a selective extraction of >74% cannabinoids, with the balance being flavonoids and terpenes. No waxes, resins or other undesired compounds are detected.

### Example 2—Marijuana Plant Material 1

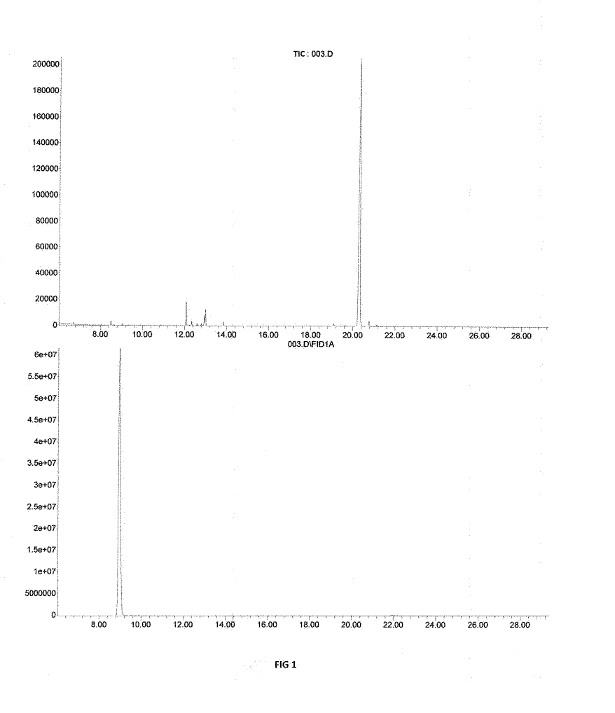

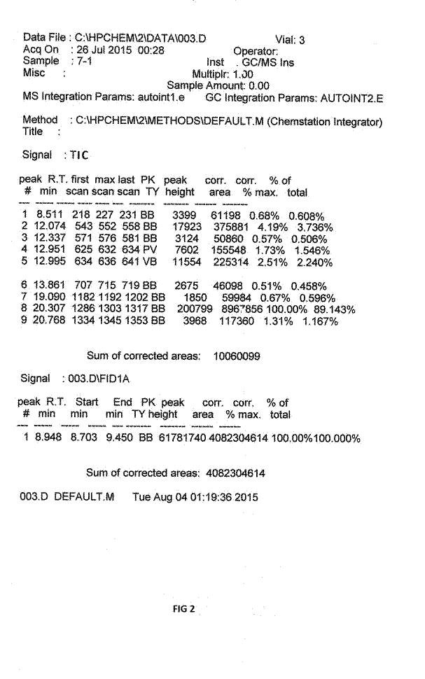

Using a method similar as described above in Example 1, 52.8 grams of a marijuana plant material gives 10.0 grams of a red oil (18.9% total yield; sample 7-1). As is, the extract, red oil is subjected to GC/MS analysis (FIG. 1 and FIG. 2). As can be seen, THC accounts for 89% of the red oil (FIG. 1 and FIG. 2, peak 8).

### Example 3—Marijuana Plant Material 2

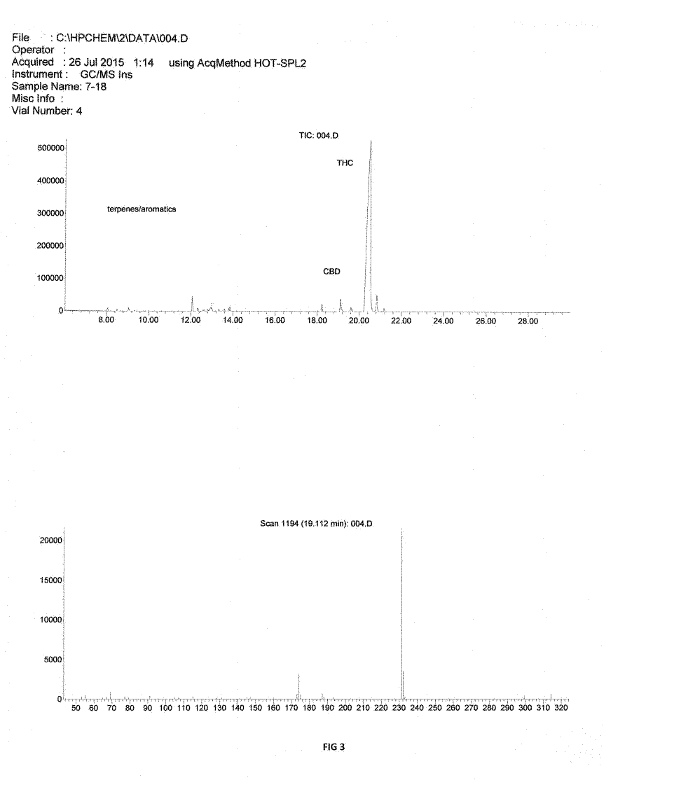

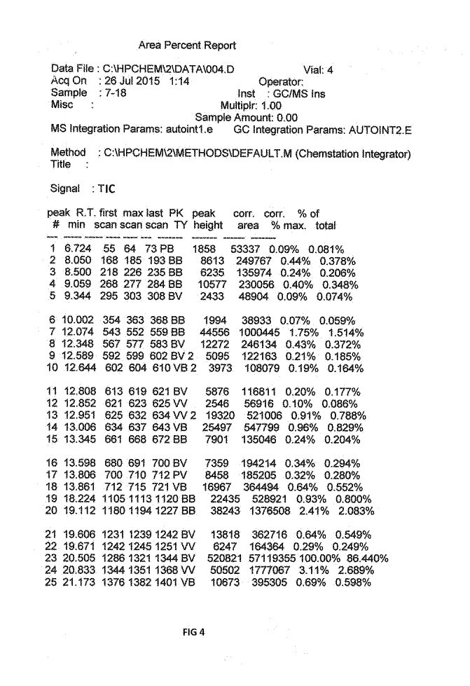

Using a method similar as described above in Example 1, 28.0 grams of a marijuana plant material gives 5.4 grams of an oil (19.3% total yield; sample 7-18). As is, the extract oil is subjected to GC/MS analysis (FIG. 3 and FIG. 4). As can be seen, THC accounts for 86.4% of the oil (FIG. 3 and FIG. 4, peak 23), with about 2.1% of the oil being CBD (FIG. 3 and FIG. 4, peak 20).

### Example 4—Wild Hemp

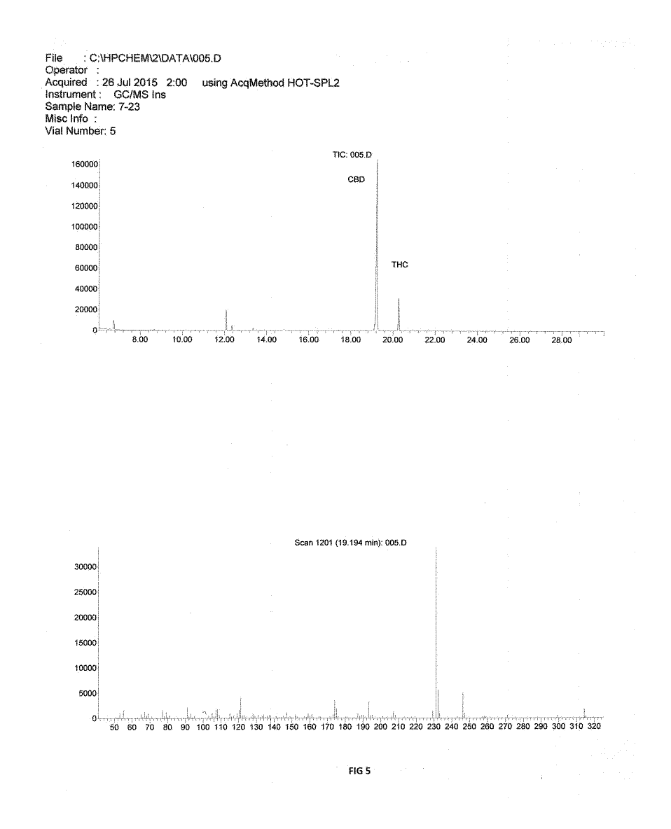

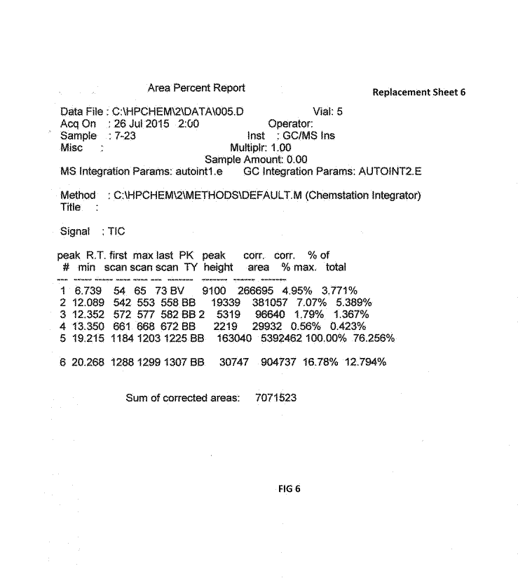

Using a method similar as described above in Example 1, 3,170.0 grams of a wild hemp plant material gives 32.0 grams of an oil (1.0% total yield; sample 7-23). As is, the extract oil is subjected to GC/MS analysis (FIG. 5 and FIG. 6). As can be seen, CBD accounts for about 76.3% of the oil (FIG. 5 and FIG. 6, peak 5), with about 12.8% of the oil being THC (FIG. 5 and FIG. 6, peak 6).

### Example 5—Marijuana Plant Material 3 (Purity Testing for Residual Solvents)

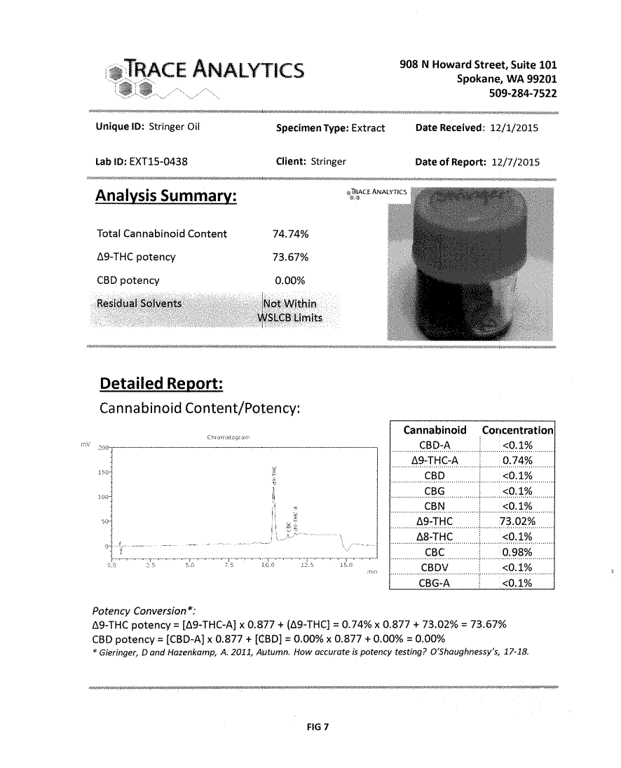

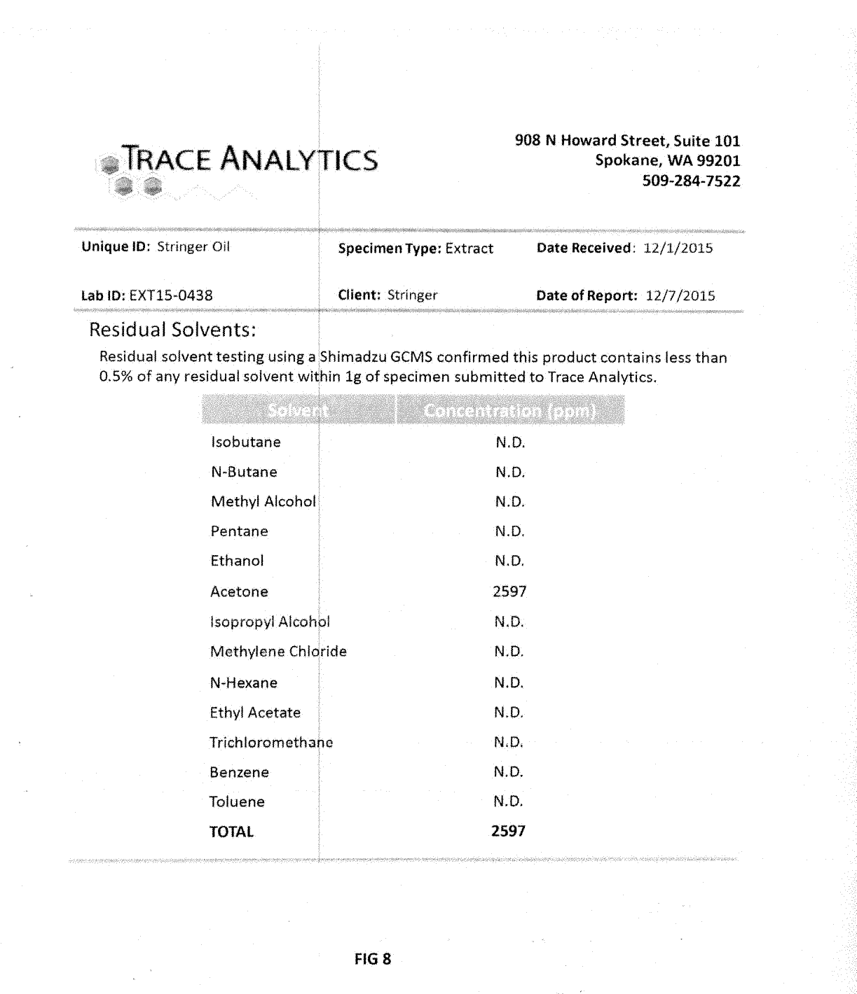

Using a method similar as described above in Example 1, 28.0 grams of a marijuana plant material gives 5.3 grams of an oil (18.9% total yield; sample EXT15-0438). As is, the extract oil is subjected to GC analysis (FIG. 7) (Trace Analytics, Spokane, Wash.). As can be seen, THC accounts for about 73.7% of the oil (FIG. 7), with the total cannabinoid content being about 74.7%. Residual solvent testing using GC/MS reveals that the extract contains less than 0.5% of any residual solvent (FIG. 8), demonstrating the high level of purity obtained by the method disclosed herein.

### Example 6—Marijuana Plant Material 4 (Purity Testing for Pesticide and Residual Solvent)

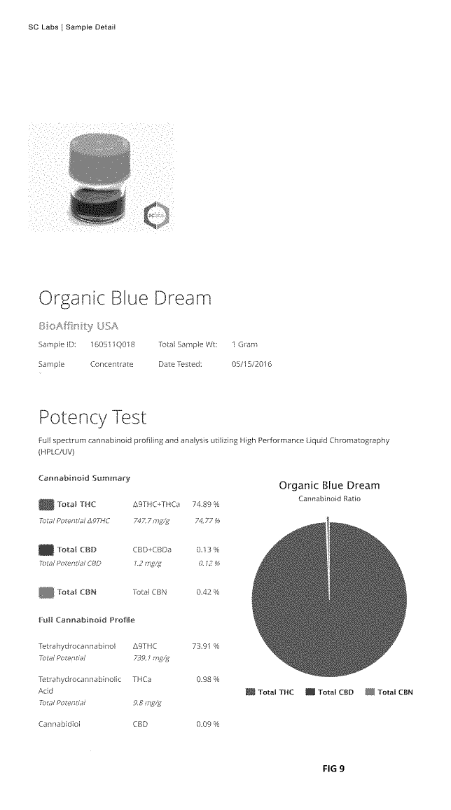

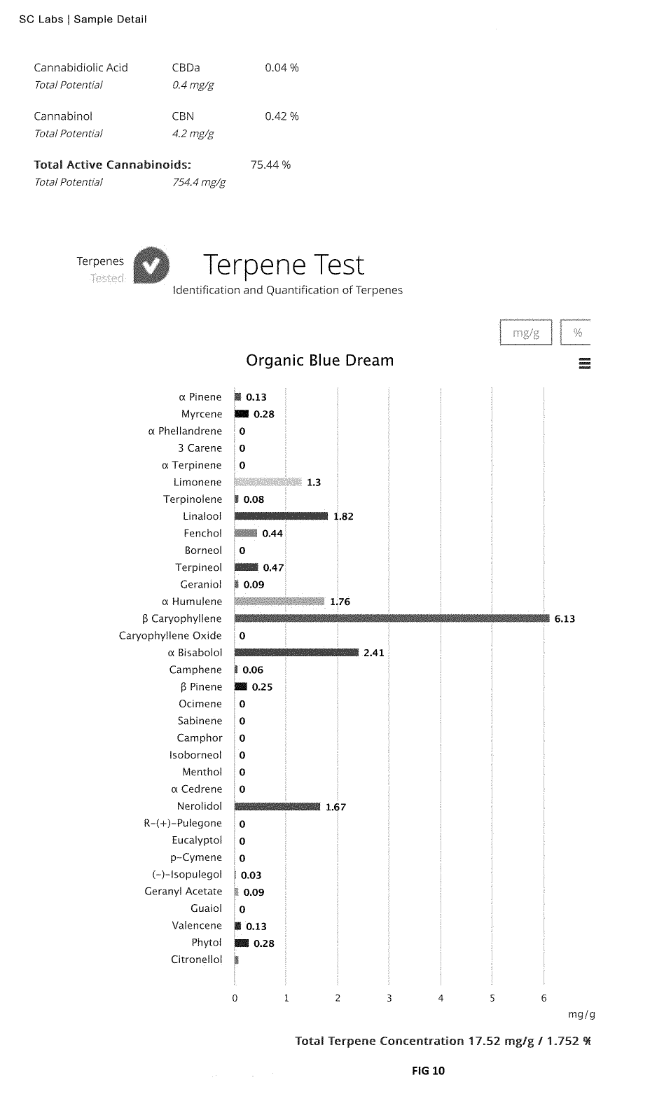

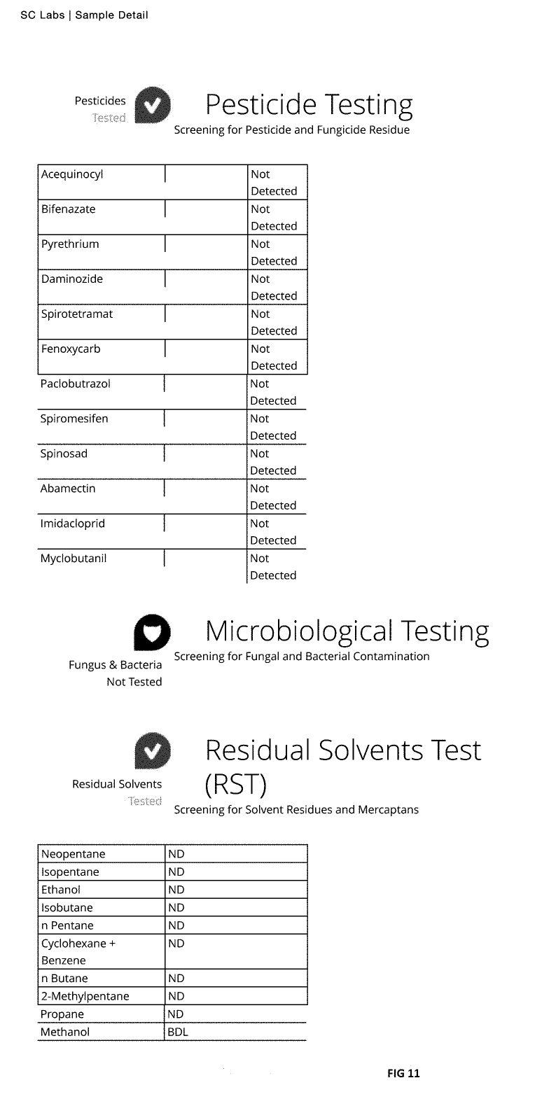

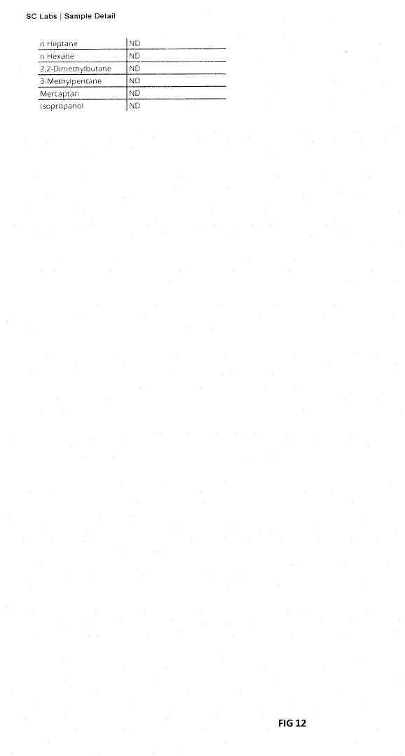

Using a method similar as described above in Example 1, 28.3 grams of a marijuana plant material gives 5.1 grams of an oil (18.0% total yield; sample Organic Blue Dream). As is, the extract oil is subjected to GC-UV analysis (FIG. 9) (SC Labs, Santa Ana, Calif.). As can be seen, THC accounts for about 74.9% of the oil (FIG. 9), with the total cannabinoid content being about 75.4%. In addition, terpene content accounts for most of the rest of the extract (FIG. 10). Finally, pesticide and solvent testing reveals that the extract contains no detectable amount of either pesticide or solvent (FIG. 11 and FIG. 12).

### Example 7—(Effect of Time on Extraction)

Extraction times exceeding 30 minutes do not produce higher yields. Time reduction, i.e. less than 30 minutes, is performed on lower quality marijuana plant material to determine effects on yields. As can be seen in Table 1 below, reducing the extraction time from 30 minutes to 5 minutes reduces the overall yield slightly. The same plant material is used throughout.

TABLE 1

| Amount of Plant | Time of | Amount of | % Overall |
|---|---|---|---|
| Material | Extraction | Extract | Yield |
| 28.3 g | 5 Minutes | 1.3 g | 4.6% |
| 28.3 g | 30 Minutes | 1.5 g | 5.3% |

It is noted that using a lower quality material provides for lower overall yields of extract, generally. As can be seen from Table 1, a slight increase in yield is afforded upon extending the extraction time from 5 minutes to 30 minutes. In other words, the process disclosed herein allows the operator to extract large amounts of material very quickly with small portable equipment. In addition, the extraction method as disclosed affords a pure product, with no purification step necessary. It is noted that no pressure need be applied to the extraction method. In short, the deficiencies of the prior art are overcome by the extraction method disclosed herein.

What is claimed is:

1. A process for preparing an extract from a plant material comprising treating the plant material with at least one polar solvent and carbon dioxide as co-solvent at a temperature of between −25° C. to −100° C. at atmospheric pressure.

2. The process according to claim 1, wherein the extract is extracted from a Cannabis genus of plant.

3. The process according to claim 1, wherein the extract is extracted from a tobacco plant.

4. The process according to claim 2, wherein the extract comprises >90% cannabinoids.

5. The process according to claim 3, wherein the extract comprises nicotine.

6. The process according to claim 1, wherein the at least one polar solvent is selected from the group consisting of ethanol, methanol, isopropanol, methyl ethyl ketone, acetone and acetonitrile.

7. The process according to claim 6, wherein the at least one polar solvent is acetone.

8. The process according to claim 1, wherein the at least one polar solvent is present in an amount of about 50% w/w relative to the carbon dioxide, and the carbon dioxide is present in an amount of about 50% w/w relative to the at least one polar solvent.

9. The process according to claim 1, wherein the at least one polar solvent is present in an amount of about 50% v/v relative to the carbon dioxide, and the carbon dioxide is present in an amount of about 50% v/v relative to the at least one polar solvent.

10. The process according to claim 4, wherein the extract is further subjected to a decarboxylation step, the >90% cannabinoids comprising cannabinoid acids, the decarboxylation step converting the cannabinoid acids to free cannabinoids.

11. The process according to claim 1, wherein the plant material is first macerated before being treated with the at least one polar solvent and the carbon dioxide.

12. The process according to claim 4, wherein the extract is afforded an overall yield of about from between 18-20%.

13. The process according to claim 4, wherein the extract consists essentially of cannabinoids, flavonoids and terpenes.

14. The process according to claim 1, wherein the treating of the plant material with the at least one polar solvent and the carbon dioxide co-solvent is from 5-30 minutes.
